(19) United States
(12) Patent Application Publication
(10) Pub. No.: US 2013/0211337 A1
(43) Pub. Date: Aug. 15, 2013

(54) INJECTION DEVICE HAVING A TWO-PART PLUNGER ROD

(71) Applicant: Verena Hofmann, Frankfurt Am Main (DE)

(72) Inventor: Verena Hofmann, Frankfurt Am Main (DE)

(73) Assignee: SANOFI-AVENTIS DEUTSCHLADN GMBH, Frankfurt am Main (DE)

(21) Appl. No.: 13/878,466

(22) PCT Filed: Oct. 25, 2011
(86) PCT No.: PCT/EP2011/068593

§ 371 Date: Apr. 9, 2013

(30) Foreign Application Priority Data
Oct. 25, 2010 (EP) 10188656.2

(51) Int. Cl. A61M 5/32

(52) U.S. Cl. CPC A61M 5/3221
USPC 604/197

(57) ABSTRACT

An injection device comprises a housing, a plunger rod and an injection needle that is movably arranged within the housing. The plunger rod is separated into a first plunger rod part and a second plunger rod part. The injection needle is movable from a first position (I) to a second position (II) by actuating the first plunger rod part The injection needle is retained within the housing in the first position (I) and projects from the housing in the second position (II). The second plunger rod partcouples to the first plunger rod part when the injection needle reaches the second position (II).

### CROSS REFERENCE TO RELATED APPLICATIONS

The present application is a U.S. National Phase Application pursuant to 35 U.S.C. §371 of International Application No. PCT/EP2011/068593 filed Oct. 25, 2011, which claims priority to European Patent Application No. 10188656.2 filed Oct. 25, 2010. The entire disclosure contents of these applications are herewith incorporated by reference into the present application.

### TECHNICAL FIELD

The present invention relates to an injection device comprising an injection needle. The injection device is adapted to avoid accidental needle stick injuries before, during and after an injection of a medication or drug contained in a cartridge. The injection device is easy and safe to use and is well suited for a self-administrated injection or for an injection administered by a health-care professional.

### BACKGROUND

Typically, injection devices that are used in combination with cartridges containing a medication are re-usable devices. The cartridge comprises a septum that is punctured by a double-ended injection needle of the injection device. Usually, the injection needle is detached from the injection device after the injection has been carried out to prevent accidental needle stick injuries.

However, the manual detachment of the injection needle puts the user of the injection device at risk of an inadvertent needle stick injury.

Thus, there is a need for an injection device that may be disposed with the cartridge and automatically provides needle safety after the injection has been completed.

### SUMMARY

It is an object of the present invention to provide an improved injection device that minimizes the risk of an accidental needle stick injury.

The object is achieved by an injection device according to claim 1.

Preferred embodiments of the invention are given in the dependent claims.

In the context of this specification, the terms distal and proximal are defined from the point of view of a person performing an injection. Consequently, a distal direction refers to a direction pointing towards the body of a patient receiving an injection and a distal end defines an end of an element that is directed towards the body of the patient. Respectively, the proximal end of an element or the proximal direction is directed away from the body of the patient receiving the injection and opposite to the distal end or distal direction.

An injection device comprises a housing, a plunger rod and an injection needle that is movably arranged within the housing. The plunger rod is separated into a first plunger rod part and a second plunger rod part. The injection needle is movable from a first position to a second position by actuating the first plunger rod part. The injection needle is retained within the housing in the first position and projects from the housing in the second position. The second plunger rod part couples to the first plunger rod part when the injection needle reaches the second position.

The injection needle in the first position is safely retained within the housing before use of the injection device to prevent inadvertent needle stick injuries. The injection needle is moved to a second position by moving the first plunger rod part in a distal direction. The second plunger rod part couples to the first plunger rod part, so that both the second plunger rod part may be moved by pushing the first plunger rod part further in the distal direction, whereby a medication is expelled through the injection needle. The first plunger rod part is actuated and moved in a single linear movement to expose the injection needle and to dispose the medication beneath the skin of a patient.

The injection device is easy to handle and is thus suitable for both a self-administered injection or for an injection administered by a healthcare professional. In particular, the injection device is adapted to deliver a vaccine beneath the skin of the patient.

The second plunger rod part may comprise a locking detent that latches to a locking recess of the first plunger rod part to couple the first plunger rod part to the second plunger rod part. The second plunger rod part remains static with respect to the housing while the first plunger rod part moves distally to expose the injection needle. The locking detent automatically latches to the locking recess when the injection needle reaches the first position. An additional interaction of the user of the injection device is not necessary.

According to a possible embodiment of the invention, the second plunger rod part coupled to the first plunger rod part is moveable by actuating the first plunger rod part. Both the exposure of the injection needle and the expelling of the medication may be performed by manually pushing the first plunger rod part distally in a single linear stroke.

An inner body may comprise a spring means that is connected to the second plunger rod part. The spring means may bias the second plunger rod part in a proximal direction. The spring means provides a biasing element that allows for a retraction of the injection needle after an injection has been performed. Thus, the spring means is an essential part to provide needle safety after a single use of the injection device.

According to a possible embodiment of the invention, the inner body is made from at least two plastics materials of different flexibility by injection moulding. The injection device comprises only a few parts preferably made from plastics materials to cut down production costs.

According to another possible embodiment of the invention, the spring means comprises a flexible sleeve that engages a tapered inner surface of the housing, whereby the flexible sleeve is resiliently and radial outwardly deflected, whereby the inner body is biased in the proximal direction.

The inner body is releasably retained in an initial position, wherein the flexible sleeve is partially stressed and a spacer means prevents a proximal movement of the inner body. The spacer means keeps the inner body initially in the initial position, in which the inner body may be moved in the distal direction. After the injection, the spacer means allow for a retraction of the inner body with respect to the housing, wherein a subsequent use of the injection device is prevented. Thus, an inadvertent needle stick injury with a contaminated injection needle is avoided.

The spacer means comprises a spherical element to releasably retain the inner body in the initial position against the biasing force of the partially stressed flexible sleeve. The spherical element automatically leaves its position to allow for a retraction of the inner body towards an end position. Additionally, a biasing element may bias the spherical element, so that the spherical element leaves its initial position as soon as the inner body moves with respect to the housing in the distal direction.

The inner body is permanently locked in the end position, wherein the inner body is retained in a proximal position. A reuse of the injection device is thus prevented to minimize the risk of an accidental needle stick injury. Furthermore, the injection device may be disposed together with the cartridge retained therein after a single injection has been carried out.

A compression spring couples the movement of the first plunger rod part to a movement of the injection needle. Thus, the injection needle may be moved towards the first position to expose the injection needle by actuating the first plunger rod part. The exposure of the injection needle, the piercing of a septum of the cartridge and the expelling of medication contained in the cartridge are achieved by a single actuation of the first plunger rod part. The injection device is thus simple to handle even for inexperienced users.

According to another possible embodiment of the invention, a limiter disc is formed to the injection needle to limit a distal movement of the injection needle with respect to the housing. The injection needle projects from the housing by a length that allows for an insertion of the injection needle into the skin of the patient at a desired penetration depth suitable for the injection.

The injection needle is surrounded by the housing when the inner body is in the initial position and/or the end position. Thus, the injection device in particular provides needle safety before and after an injection is performed.

According to yet another possible embodiment, the injection needle comprises a proximal end adapted to pierce a septum of a cartridge containing a medication. The injection device is adapted to be used in combination with cartridges sealed by a septum, which are widely used in medical applications.

Alternatively, a pre-filled syringe comprising a needle may be inserted in the injection device.

The medication may be expelled through the injection needle by a movement of the second plunger rod part in the distal direction. The second plunger part may be connected to a piston fluid-tightly sealing a proximal end of the cartridge or the pre-filled syringe retained in the injection device. The medication is conveniently injected by a user pushing the first plunger rod part distally in a single stroke.

Further scope of applicability of the present invention will become apparent from the detailed description given hereinafter. However, it should be understood that the detailed description and specific examples, while indicating possible embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will be better understood from the detailed description given in the following. The accompanying drawings are given for illustrative purposes only and do not limit the scope of the present invention.

FIG. 1 shows a sectional view of the injection device according to a first embodiment of the invention.

FIGS. 2A to 2C schematically illustrate an injection carried out with the injection device.

FIG. 3 shows a perspective view of the injection device according to a second embodiment of the invention.

FIG. 4 shows a sectional view of the injection device according to a second embodiment of the invention.

Corresponding parts are marked with the same reference symbols in all figures.

### DETAILED DESCRIPTION

FIG. 1 shows an injection device 1 comprising a housing 2 and a plunger rod 3 according to a first embodiment of the invention. The plunger rod 3 comprises a hollow first plunger rod part 3.1 with an axial length that exceeds an axial length of a second plunger rod part 3.2. The second plunger rod part 3.2 is inserted in the hollow first plunger rod part 3.1 and is connected to a piston 4 sealing a proximal end of a cartridge 5 containing a medication or drug.

The second plunger rod part 3.2 may be coupled to the first plunger rod part 3.1 by a locking detent 3.2.1 that latches to a locking recess 3.1.1 of the first plunger rod part 3.1.

The housing 2 comprises two outwardly protruding gripping wings 2.1 that support the fingers of a user in carrying out an injection. The housing 2 further comprises a tapered inner surface 2.2 and an inner sleeve 2.3.

An injection needle 6 is movably arranged within the inner sleeve 2.3. A radial protruding limiter disc 6.1 is moulded to the injection needle 6 that is made from a plastics material. The limiter disc 6.1 is inserted into the inner sleeve 2.3. The inner sleeve 2.3 guides the movement of the limiter disc 6.1 and the injection needle 6 connected to the limiter disc 6.1 from a first position I to a second position II. The injection needle 6 is surrounded by the housing 2 in the first position I, whereas the injection needle 6 projects distally from the housing 2 in the second position II. In the second position II, the limiter disc 6.1 abuts a distal end wall 2.4 of the housing 2, so that a distal movement of the injection needle 6 with respect to the housing 2 is limited.

A compression spring 7 is arranged within the inner sleeve 2.4. A proximal end of the compression spring 7 is connected to the hollow first plunger rod part 3.1, whereas a distal end of the compression spring 7 is connected to the limiter disc 6.1.

A distal end of the first plunger rod part 3.1 is received in a proximal end of the inner sleeve 2.3. The compression spring 7 couples the movement of the first plunger rod part 3.1 to the movement of the injection needle 6, so that the injection needle 6 may be moved from the first position I to the second position II by pushing the first plunger rod part 3.1 in the distal direction.

The cartridge 5 containing the medication is received within the inner sleeve 2.3. A puncturable septum 5.1 fluid-tightly seals a distal end of the cartridge 5. The injection needle 6 comprises a proximal end that protrudes the limiter disc 6.1 in the proximal direction. The proximal end of the injection needle 6 is adapted to puncture the septum 5.1.

An inner body 8 is arranged within the housing 2. According to the first embodiment, the inner body 8 comprising a spring means S is connected to the second plunger rod part 3.2. The inner body 8 may be made from two plastics materials of different flexibility in particular by injection moulding. The spring means S constitutes a substantially cylindrical flexible sleeve 8.1 of the inner body 8. The flexible sleeve 8.1 is made from a resilient plastics material like, for example, an elastomer. The inner body 8 shown in FIG. 1 is retained in an initial position PI, whereby the flexible sleeve 8.1 engages the tapered inner surface 2.2. The flexible sleeve 8.1 is partially stressed and slightly deflected in a radial outward direction, so that the inner body 8 is biased in the proximal direction.

Alternatively, the spring means S may constitute a plurality of flexible arms (not illustrated) formed to opposite sides of the inner body 8. The flexible arms are made from a resilient plastics material like an elastomer and engage the tapered surface 2.2, whereby the flexible arms are radial outwardly deflected to bias the inner body 8 with respect to the housing 2 in the proximal direction.

In an alternative embodiment, the spring means S constitutes a second compression spring that has an inner diameter that is sized in a manner that the first plunger rod part 3.1 may be inserted in the second compression spring. The second compression spring may bear against the inner body 8 and against the distal end wall 2.4 to bias the inner body 8 in the proximal direction.

A spacer means 9 limits a proximal movement of the inner body 8 with respect to the housing 2 before the injection. The spacer means 9 comprises a resiliently deflectable rib 9.1 that protrudes from an inner surface of the housing 2 in a radial inward direction.

A spherical element 9.2 is clamped between the rib 9.1 and a proximal surface 8.2 of the inner body 8. The spherical element 9.2 is made from a plastics material and keeps the inner body 8 in the initial position PI, wherein the inner body 8.2 is movable in the distal direction.

Additionally or alternatively, the spherical element 9.2 may be connected to a biasing element 9.3 that ensures that the spherical element 9.2 leaves its position between the rib 9.1 and a proximal surface 8.2 as soon as the inner body 8 is moved in the distal direction.

A locking notch 8.3 is formed to the inner body 8. The radial inwardly protruding rib 9.1 latches to the locking notch 8.3 after a single injection has been performed. The rib 9.1 engaging the locking notch 8.3 retains and irreversibly locks the inner body 8 in an end position PII, in which the inner body 8 is retracted in the proximal direction with respect to the housing 2. As the movement of the injection needle 6 is coupled to the movement of the inner body 8 by the compression spring 7 and the first plunger rod part 3.1 locked to the second plunger rod part 3.2, the injection needle 6 is retained in the retracted first position I when the inner body 8 is in the end position PII.

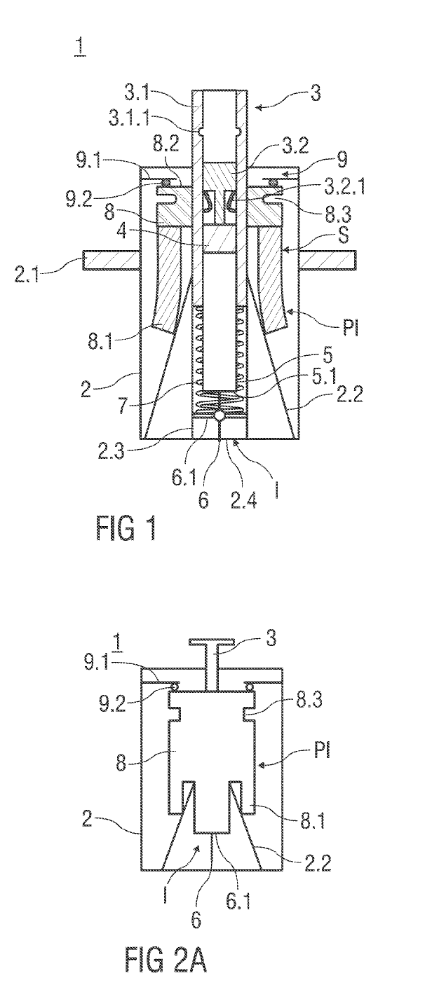

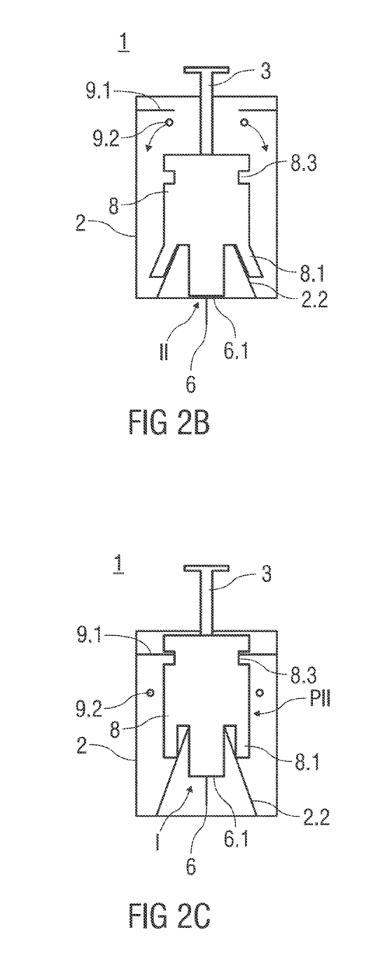

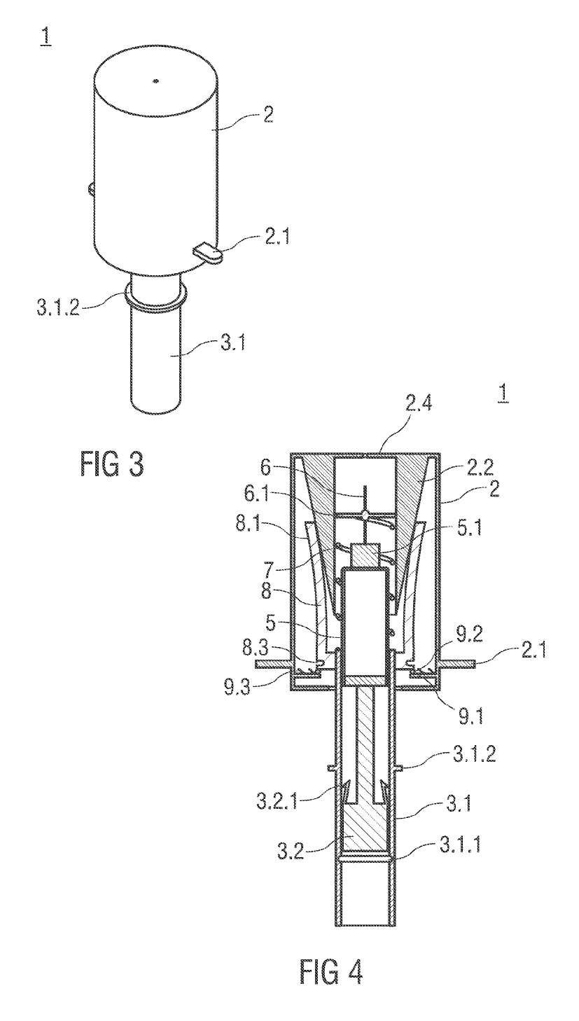

FIGS. 2A to C schematically illustrate an injection carried out with the injection device 1. The injection is carried out as follows:

The first plunger rod part 3.1 is manually pushed in the distal direction to move the injection needle 6 from the first position I to the second position II, as indicated in FIG. 2A and B. In the second position II, the limiter disc 6.1 abuts the distal end wall 2.4 and the injection needle 6 projects distally from the housing 2 by a length that may correspond to a penetration depth of the injection needle 6.

When the injection needle 6 reaches the second position II, the locking detent 3.2.1 latches to the locking recess 3.1.1, so that the second plunger rod part 3.2 may be moved by actuating the first plunger rod part 3.1 protruding the housing 2 in the distal direction. The first plunger rod part 3.1 is actuated, whereby the first and the second plunger rod part 3.1, 3.2 jointly move in the distal direction. The cartridge 5 is pushed distally by the second plunger rod part 3.2 until the proximal end of the injection needle 6 pierces the septum 5.1 of the cartridge 5. The injection needle 6 is now in fluid communication with an interior of the cartridge 5 and the compression spring 7 is compressed and energized. The injection device 1 is dimensioned in a manner that the injection needle 6 protrudes into an interior of the cartridge 5 by a length that allows for a complete depletion of the cartridge 5.

As the second plunger rod part 3.2 is connected to the inner body 8, the inner body 8 simultaneously moves in the distal direction, whereby the spherical element 9.2 leaves its position between the proximal surface 8.2 and the rib 9.1. From now on, the inner body 8 is allowed to move proximally with respect to the housing 2 when the first plunger rod part 3.2 is released.

The injection needle 6 is inserted into the skin of the patient. The first and the second plunger rod part 3.1, 3.2 are moved further in the distal direction, whereby the piston 4 moves distally to expel the drug or medication contained in the cartridge 5 beneath the skin of the patient.

At the same time, the inner body 8 connected to the second plunger rod part 3.2 moves distally, whereby the flexible sleeve 8.1 moves along the tapered surface 2.2 of the housing 2. The flexible sleeve 8.1 is deflected in the radial outward direction, so that the inner body 8 is strongly biased in the proximal direction when the piston 4 reaches a distal end of the cartridge 5 at the end of the injection stroke.

The injection device 1 is removed from the injection site. The stressed flexible sleeve 8.1 relaxes, whereby the inner body 8 is moved in the proximal direction past the inwardly protruding rib 9.1. The rib 9.1 is radial deflected until it latches to the locking notch 3.2.1, whereby the inner body 8 is locked to the end position PII. The compression spring 7 connected to the first plunger rod part 3.1 and the limiter disc 6.1 moves the injection needle 6 back to the first position I, so that a needle stick injury may be avoided.

FIG. 4 shows a perspective view of the injection device 1 according to a second embodiment of the invention. A circumferential and radial outwardly protruding ring 3.1.2 is formed to an outer surface of the first plunger rod part 3.1.

FIG. 5 shows a sectional view of the injection device 1 according to the second embodiment. According to the second embodiment, the second plunger rod part 3.2 is not connected to the inner body 8. Instead the ring 3.1.2 is adapted to abut the inner body 8 to couple the movement of the first plunger rod part 3.1 to the inner body 8.

When the first plunger rod part 3.1 is pushed distally to move the injection needle 6 from the first position I to the second position II, the ring 3.1.2 slides into the housing 2. The ring 3.1.2 abuts the inner body 8, so that the inner body 8 and the flexible sleeve 8.1 integrated to the inner body 8 may be moved in the distal direction by pushing the first plunger rod part 3.1 further in the distal direction. The flexible sleeve 8.1 engages the tapered inner surface 2.2, whereby the flexible sleeve 8.1 is deflected in the radial outward direction to bias the inner body 8 in the proximal direction at the end of the injection stroke. The spherical element 9.2 that is frictionally held in position between the proximal surface 8.2 and the rib 9.1 is retracted by the action of the relaxing biasing element 9.3. The inner body 8 is allowed to move proximally with respect to the housing 2 when the first plunger rod part 3.1 is released.

After the injection device 1 is removed from the injection site, the stressed flexible sleeve 8.1 relaxes, whereby the inner body 8 is moved in the proximal direction past the inwardly protruding rib 9.1. The rib 9.1 engages the locking notch 3.2.1 to lock the inner body 8 to the end position PII. As the ring 3.1.2 abuts the inner body 8, the first plunger rod part 3.1 jointly moves with the inner body 8 in the proximal direction, whereby the injection needle 6 is moved back to the first position I and retracted into the housing 2.

1-14. (canceled)

15: An injection device comprising:
a housing,
a plunger rod and
an injection needle that is movably arranged within the housing, wherein the plunger rod is separated into a first plunger rod part and a second plunger rod part and the injection needle is movable from a first position (I) to a second position (II) by actuating the first plunger rod part, wherein the injection needle is retained within the housing in the first position (I) and projects from the housing in the second position (II), and wherein the second plunger rod part couples to the first plunger rod part when the injection needle reaches the second position (II).

16: An injection device according to claim 15, characterized in that the second plunger rod part comprises a locking detent that latches to a locking recess of the first plunger rod part to couple the first plunger rod part to the second plunger rod part (3.2).

17: An injection device according to claim 15, characterized that the second plunger rod part coupled to the first plunger rod part is moveable by actuating the first plunger rod part.

18: An injection device according to claim 15, characterized in that an inner body comprising a spring means (S) is connected to the second plunger rod part, wherein the spring means (S) biases the second plunger rod part in a proximal direction.

19: An injection device according to claim 18, characterized in that the inner body is made from at least two plastics materials of different flexibility by injection moulding.

20: An injection device according to claim 18, characterized in that the spring means (S) comprises a flexible sleeve that engages a tapered inner surface of the housing, whereby the flexible sleeve is resiliently and radial outwardly deflected.

21: An injection device according to claim 20, characterized in that the inner body is releasably retained in an initial position (PI), wherein the flexible sleeve is partially stressed and a spacer means prevents a proximal movement of the inner body.

22: An injection device according to claim 21, characterized in that the spacer means comprises a spherical element.

23: An injection device according to claim 15, characterized in that inner body is permanently locked in an end position (PII), wherein the inner body is retained in a proximal position.

24: An injection device according to claim 18, characterized in that the injection needle is surrounded by the housing when the inner body is in the initial position (PI) and/or the end position (PII).

25: An injection device according to claim 15, characterized in that a compression spring couples the movement of the first plunger rod part to a movement of the injection needle.

26: An injection device according to claim 15, characterized in that a limiter disc is formed to the injection needle to limit a distal movement of the injection needle with respect to the housing.

27: An injection device according to claim 15, characterized in that the injection needle comprises a proximal end adapted to pierce a septum of a cartridge containing a medication.

28: An injection device according to claim 27, characterized in that the medication is expelled through the injection needle by a movement of the second plunger rod part in the distal direction.
